(19) United States
(12) Patent Application Publication
(10) Pub. No.: US 2021/0346564 A1
(43) Pub. Date: Nov. 11, 2021

(54) INLET-OUTLET MICROBE SAFETY SYSTEM

(71) Applicant: Neil Robert Jetter, Palm Beach Gardens, FL (US)

(72) Inventor: Neil Robert Jetter, Palm Beach Gardens, FL (US)

(21) Appl. No.: 17/202,844

(22) Filed: Mar. 16, 2021

Related U.S. Application Data
(60) Provisional application No. 62/990,056, filed on Mar. 16, 2020.

(51) Int. Cl. A61L 9/22; A62B 18/08; A61L 9/20; A41D 13/11

(52) U.S. Cl. CPC A61L 9/22; A62B 18/08; A61L 2209/12; A41D 13/11; A61L 9/20

(57) ABSTRACT

An inlet-outlet microbe safety (IOMS) system includes a face mask configured for being secured over at least a nose and a mouth of an individual that is further configured to receive input gas and to output waste gas exhaled by the individual. A breathing device assembly includes a first microbe killer device in a path of the input gas and a second microbe killer device in a path of the waste gas. The face mask is coupled by tubing to the breathing device assembly.

### CROSS REFERENCE TO RELATED APPLICATIONS

This application claims the benefit of Provisional Application Ser. No. 62/990,056 entitled “MICROBE SAFETY DEVICE”, filed Mar. 16, 2020, which is herein incorporated by reference in its entirety.

### FIELD

This Disclosure relates to microbe safety devices.

### BACKGROUND

Microorganisms, or microbes which is the term used herein, can cause disease, and generally come in a variety of different forms. Viruses and bacteria are microbes familiar to most because they are publicized as the principal source of infectious disease. However, fungi, protozoa, and helminths can also cause infectious diseases.

Regarding a virus, there are several mechanisms that all known to be needed to be present for a viral-based disease to develop in an individual.
1. Implantation of the virus at a portal of entry of a host (or individual). The virus must implant at an entry portal in the individual, such as the nose, the eyes, or the mouth of the individual. Implantation is the first stage of pathogenesis. The implantation frequency is known to be at its highest when viruses directly contact living cells. A common source for entry is known to be airborne contact from a sneeze or cough from a virus-infected individual. It is known that the average sneeze or cough propels around 100,000 contagious germs into the air at speeds up to about 100 miles per hour. These germs can carry viruses, such as influenza, respiratory syncytial virus (RSV) and adenoviruses, that can cause the common cold, and more recently COVID-19 known as the “coronavirus” and its variants.
2. Local replication and local spread of the virus. local replication and spread of the virus follows the implantation of the virus (step 1). Replicated virus from the initially infected cell has the ability to disperse to adjacent extracellular fluids or cells. Spread of the virus occurs by the neighboring cell being infected, or the virus being released into extracellular fluid. The invading virus reproduces itself in large numbers. The invading virus typically accomplishes reproduction intracellularly.
3. Dispersal of the virus. The replicated viruses spread to target organs (disease sites) throughout the body of the host. The most common route of spread of the virus from the portal of entry is the circulatory system, which the virus reaches via the lymphatic system. Viruses can access target organs from the blood capillaries by multiplying inside endothelial cells, moving through gaps, or by being carried inside the organ on leukocytes.
4. Shedding is the final step. The viruses spread to sites where shedding into the environment occurs to repeat the implantation step 1 described above. The respiratory, alimentary and urogenital tracts and the blood are known to be the most common sites of shedding.

### SUMMARY

This Summary is provided to introduce a brief selection of disclosed concepts in a simplified form that are further described below in the Detailed Description including the drawings provided. This Summary is not intended to limit the claimed subject matter's scope.

Disclosed aspects include an inlet-outlet microbe safety (IOMS) system that can be a portable device includes a face mask configured for being secured over at least a nose and a mouth of an individual that is further configured to receive input gas and to output waste gas exhaled by the individual. A breathing device assembly includes a first microbe killer device in a path of the input gas and a second microbe killer device in a path of the waste gas. The face mask is coupled by tubing to the breathing device assembly. The first microbe killer device is provided by disclosed IOMS prevents microbes such as viruses from implanting at the nose or the mouth (optionally also at the eyes) of the individual. The second microbe killer device positioned in the outlet path of a waste gas stream exhaled by the individual kills the microbes in the case the individual is infected with one or more microbes.

### BRIEF DESCRIPTION OF THE DRAWINGS

FIG. 1 is a schematic perspective view of an example IOMS system that is generally portable for being utilized by a movable individual, according to an example aspect.

FIG. 2 is a cross-sectional view of an example DBD plasma reactor that can be used as a microbe killer device for disclosed aspects.

### DETAILED DESCRIPTION

Disclosed embodiments are described with reference to the attached figures, wherein like reference numerals, are used throughout the figures to designate similar or equivalent elements. The figures are not drawn to scale and they are provided merely to illustrate aspects disclosed herein. Several disclosed aspects are described below with reference to example applications for illustration. It should be understood that numerous specific details, relationships, and methods are set forth to provide a full understanding of the embodiments disclosed herein.

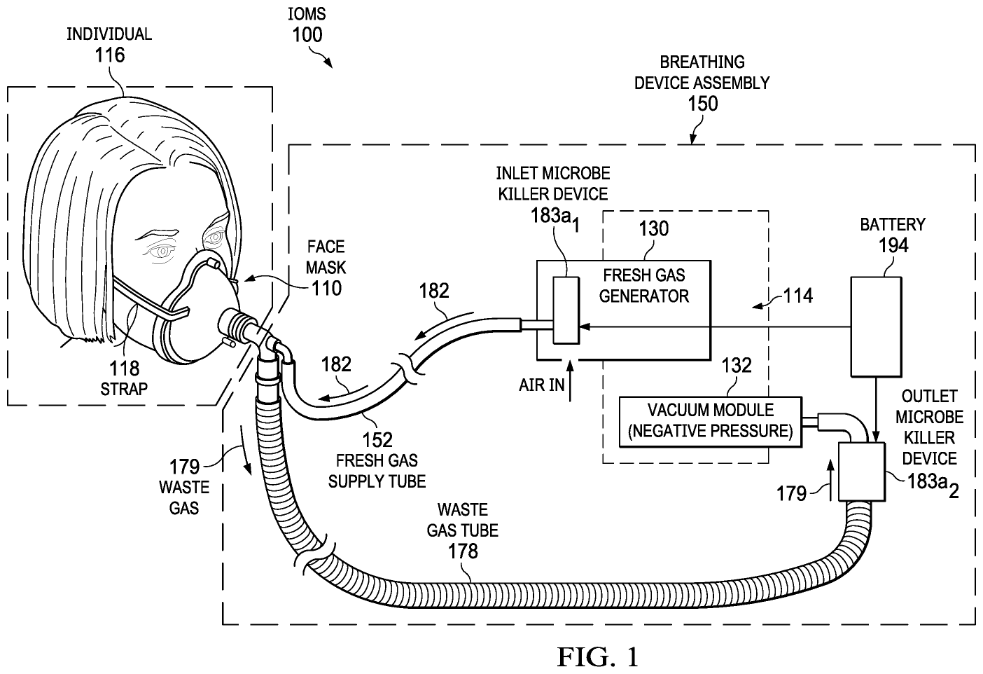

FIG. 1 is a schematic perspective view of an example of IOMS system 100 for use by an individual 116 comprising a respiratory face mask 110 coupled to a breathing device assembly 150 that includes a two-way gas device 114 that comprises a fresh gas supply module 130 for input air and a vacuum module 132 for receiving waste gas. The respiratory face mask 110 is shown optionally including a strap 118 for its securing the facemask to avoid microbes from entering inside to avoid implantation at the nose or the mouth of the individual 116, which is recognized to represent the most common portals of entry of the individual. The respiratory face mask 110 also prevents microbes from emerging outside, such in the case of a sneeze or cough by the individual 116.

The two-way gas device 114 can comprise a device used commonly in the practice of dentistry. For example, the two-way gas device 114 may be commercially available. In dentistry the gas supply module 130 is configured to supply both nitrous oxide (NO) and 02, and includes valve controls for adjusting the flow of each to produce a mixture of any desired concentration. The vacuum module 132 provides a source of negative pressure (vacuum) to draw in the waste gas 179.

The face mask 110 can be replaced by an article that covers more of the head, such as a helmet, or an article that beside the mouth and nose also covers the eyes of the individual 116. The respiratory face mask can in one arrangement comprise a surgical face mask commonly worn by medical professionals. Surgical face masks also protect the wearer's mouth, nose and mucosa from contacting splashes or sprays of the patient's blood or other body fluids, and from airborne microorganisms. Typical face masks include a body that covers the nose and mouth of the individual 116 and two sets of ties that are attached to the body that the wearer must tie behind the individual's 116 head to secure the mask to the individual's 116 face. Having two sets of ties provides the face mask 110 with some adjustability as the wearer may position the ties to suit the individual's 116 comfort and preference.

There is an inlet microbe killer device 183a1 positioned in the path of the fresh gas supply 182 that is supplied through the fresh gas supply tube 152 and an outlet microbe killer device 183a2 in the path of the waste gas 179 exhaled by the individual 116. A variety of different microbe killer devices can be used with disclosed aspects. One example of a microbe killer device is germicidal air purifiers that use materials and technologies such as based on ultraviolet (UV) light, natural silver, and sterilizing heat to eliminate airborne microbes from the air. A UV air purifier uses an internal UV-C germicidal light to kill airborne microbes such as bacteria, viruses, germs, and allergens as room air moves through the system. The UV light eliminates these microbes by breaking down their genetic structures whether DNA-based or RNA-based by damaging the DNA or the RNA, and also deactivating the microbes reproductive capabilities. Another example microbe killer device is a dielectric barrier discharge (DBD) plasma reactor which is a non-thermal reactor.

The inlet microbe killer device 183a1 associated with the fresh gas supply is configured to kill microbes received in from the ambient. For example, the recirculated cabin air in a commercial airplane can be a potentially dangerous ambient because it may include microbes from one or more infected individuals on the airplane. Disclosed IOMS may be utilized by passengers on an airplane. In this case the IOMS system 100 may include an electrical receptacle (plug) that is adapted to receive electrical power, and is wired to provide electricity including to the microbe killer devices.

The outlet microbe killer device 183a2 can kill microbes exhaled as waste gas 179 by the individual 116 that flows through a waste gas tube 178. There is a battery 194 shown, such as a rechargeable lithium battery, for generally powering light sources (e.g., UV light source) in the microbe killers 183a1 and 183a2. As noted above, a UV light-based air purifier uses an internal UV-C germicidal light to kill airborne bacteria, viruses, germs, and allergens as room or other environment air moves through the system.

There may be some passive microbe killer devices possible which do not require a battery or other power source. For example, silver nanomaterials silver (Ag) nanoparticles are known to have good microbe killing properties.

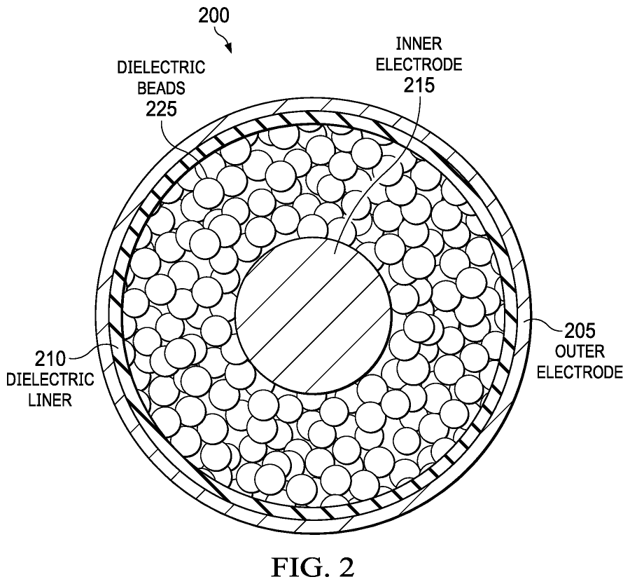

FIG. 2 is a cross-sectional view of an example DBD plasma reactor 200 that can be used as a microbe killer device for disclosed aspects. The DBD plasma reactor 200 comprises an outer electrode 205, that is radially outside a dielectric liner 210, and there is also an inner electrode 215. Between the respective electrodes there is a void region that is shown filled with dielectric beads 225, such as glass beads. In operation, the battery 194 shown in FIG. 1 supplies DC power, that is converted to alternating current (AC) power by suitable DC/AC converter, where the AC power is applied between the inner electrode 215 and the outer electrode 205 to generate the plasma. The dielectric beads 225 function by each capturing small electrical arc discharges. As AC power is applied to the DBD plasma reactor 200 results in electrons are generated and atoms are pulled from their molecules, which generally produces a silent plasma glow.

The result of operating the DBD plasma reactor is the generation of a large number free radicals, which are known to be highly reactive particles that look to reach a stable equilibrium by forming new compounds. The oxygen radical is particularly excited, and when it reacts with a molecule of oxygen gas (O₂) at room temperature which will be present with inlet and outlet gas flows, where the reaction of the O₂ with the oxygen radical rapidly forms O₃ which is known as ozone. Within a short period of time, such as about 1/10 of a second, due to the presence of a significant concentration ozone, plasma exposure can rupture a microbe's cell's wall, including viruses and bacteria, impairing and destroying the microbe's normal activity to be properly considered to be a microbe killer device.

Disclosed IOMS systems are generally configured to be worn around one's waist, such as in a holster arrangement analogous to how a smartphone is held, or held in one's pocket. It may also be possible to fit disclosed IOMS systems in one's shirt pocket or equivalent.

While various disclosed aspects have been described above, it should be understood that they have been presented by way of example only, and not as a limitation. Numerous changes to the disclosed aspects can be made in accordance with the Disclosure herein without departing from the spirit or scope of this Disclosure. Thus, the breadth and scope of this Disclosure should not be limited by any of the above-described aspects. Rather, the scope of this Disclosure should be defined in accordance with the following claims and their equivalents.

1. An inlet-outlet microbe safety (IOMS) system, comprising:
a face mask configured for being secured around at least a nose and a mouth of an individual that is further configured to receive input gas and to output waste gas exhaled by the individual, and
a breathing device assembly that includes a first microbe killer device in a path of the input gas and a second microbe killer device in a path of the waste gas,
where the face mask is coupled by tubing to the breathing device assembly.

2. The IOMS system of claim 1, where the IOMS further comprises a battery.

3. The IOMS system of claim 2, wherein the first microbe killer device and the second microbe killer device both include an ultraviolet light source that are each coupled to receive electrical power from the battery.

4. The IOMS system of claim 2, wherein the first microbe killer device and the second microbe killer device both include a dielectric barrier discharge (DBD) plasma reactor that are each coupled to receive power from the battery.

5. The IOMS system of claim 1, further comprising a two-way gas device that comprises a fresh gas supply module including the first microbe killer device for supplying the input air and a vacuum module including the second microbe killer device for receiving the waste gas.

6. A method of microbe safety for an individual, comprising:
positioning a face mask to be secured around at least a nose and a mouth of an individual, were in the facemask is configured to receive input gas and to output waste gas exhaled by the individual, and
utilizing a breathing device assembly that includes a first microbe killer device in an input path of the input gas and a second microbe killer device in an output path of the waste gas, wherein the face mask is coupled by tubing to the breathing device assembly,
wherein as the individual breathes the first microbe killer device kills microbes in the input path, and wherein the second microbe killer kills microbes in the output path when exhaled by the individual.

7. The method of claim 6, wherein the first microbe killer device and the second microbe killer device both include an ultraviolet light source that are each coupled to receive electrical power from the battery.

8. The method of claim 6, wherein the first microbe killer device and the second microbe killer device both include a dielectric barrier discharge (DBD) plasma reactor that are each coupled to receive power from a battery.
